(19) United States
(12) Patent Application Publication
(10) Pub. No.: US 2020/0406029 A1
(43) Pub. Date: Dec. 31, 2020

(54) DEVICES AND METHODS FOR STIMULATION OF HAIR GROWTH

(71) Applicant: INNOGEN TECHOLOGIES LTD., Tel Aviv (IL)

(72) Inventors: Ines VERNER RASHKOVSKY, Savyon (IL); Boris VAYNBERG, Zichron Yaakov (IL)

(21) Appl. No.: 17/022,971

(22) Filed: Sep. 16, 2020

Related U.S. Application Data
(63) Continuation of application No. 13/625,886, filed on Sep. 25, 2012.

(51) Int. Cl. A61N 1/32; A61N 1/04

(52) U.S. Cl. CPC A61N 1/32; A61N 1/322; A61B 2018/00732; A61N 1/0476; A61N 1/326; A61N 1/0464

(57) ABSTRACT

The present invention discloses a comb-like device for stimulation of hair re-growth on the epidermis of an area of skin of a subject and methods of stimulating hair re-growth for stimulation of hair re-growth.

### CROSS REFERENCE TO RELATED APPLICATIONS

This application is a Continuation of U.S. patent application Ser. No. 13/625,886 filed Sep. 25, 2012. The contents of the above application is incorporated by reference as if fully set forth herein in its entirety.

### FIELD AND BACKGROUND OF THE INVENTION

The invention, in some embodiments, relates to the field of cosmetic treatment, and more particularly, but not exclusively, to methods and devices for stimulation of hair growth.

Hair loss is very common in both men and women. In males, male pattern hair loss, may start at any age after puberty and its frequency and severity increase with advancing age.

During their teens, almost all Caucasian males develop recession of the frontal temporal hairline. Deeper recessions and vertex balding have a later onset in most males. At the age of 70, about 80% of men have more pronounced baldness. In females, female pattern hair loss is less common than male balding but as in males, may start at any age after puberty with an increase in frequency and severity with aging. At the age of 30, around 2-5% of females will have remarkable thinning of hair, rising to 40% at the age of 70. Both male and female pattern hair loss impair the quality of life and cause psychological distress that is more pronounced in females. Moreover with advancing age there is a decrease in the quantity of hair follicles, decrease in hair diameter, a slower growth rate and a higher quantity of hair follicles in the telogen growth phase in both sexes.

Different treatment modalities are currently available for the treatment of male or female pattern hair loss, none of which give satisfactory results:

### (1) Medication

For both male and female pattern hair loss, long-term use of topical minoxidil 2-5% solution can give variable results with the recurrence of hair loss shortly after stopping the medication. Minoxidil is associated with a number of side-effects, including scalp irritation or allergic contact dermatitis. Sometimes, unwanted hair growth elsewhere on the body may be seen, especially with use of the 5% solution.

Oral finasteride is a competitive 5-α-reductase inhibitor, which reduces dihydrotestosterone levels in the scalp and in serum by 70%. This medication can be used in males and can attenuate male pattern hair loss when used over a number of years. A gradual relapse occurs after discontinuation of treatment. Side effects include sexual dysfunction (in 4.2-8.7% of users) and a probable increased risk of prostate cancer.

Oral anti-androgens have been suggested for use in females but proof of their efficacy is still lacking.

### (2) Transplantation

In both males and females, hair transplantation surgery can be performed with variable results. Such procedures are invasive and expensive.

### (3) Light-Based Devices

A number of products using low level laser therapy (LLLT) for simulation of hair growth have been recently introduced and one of them (HairMax LaserComb®) has obtained FDA approval. The mechanism of action is not known and the efficacy of these LLLT products is very limited.

### SUMMARY OF THE INVENTION

The present invention relates to devices and methods comprising use of localized electrical energy to stimulate an area of hair loss, thereby promoting re-growth of hair in the area. In some embodiments, the area of hair loss is an area of the scalp. In some embodiments, a controlled wound is induced, wherein healing of the wound induces hair re-growth. In some embodiments, the device of the present invention is safe and simple to use, typically producing predictable and reproducible results, typically independent of skin type, in preferred embodiments with little or no pain.

According to some embodiments, there is provided a device for stimulation of hair re-growth on an area of skin of a subject, the device comprising an array of electrodes comprising at least one contact electrode, and at least one ground electrode, each of the electrodes configured to conduct electrical current to a surface of an area of skin via a contact surface of an electrode, wherein the electrical current delivers energy sufficient to stimulate hair growth, e.g. to cause localized heating of the area of skin, wherein the localized heating promotes re-growth of hair in the area.

In some embodiments, a localized wound is induced, wherein healing of the localized wound stimulates hair re-growth. In some embodiments, the array of electrodes comprises at least two contact electrodes and at least one ground electrode.

According to some embodiments, the device may be configured such that the function of at least one of the electrodes of the array of electrodes can be optionally switched between being a ground electrode and being a contact electrode, for example using a standard switch mechanism as known in the art.

According to some embodiments, the device is configured such that at least one pair of a first electrode and a second electrode of the array of electrodes can be switched between two states: a first state wherein the first electrode is a ground electrode and the second electrode is a contact electrode; and a second state wherein the first electrode is a contact electrode and the second electrode is a ground electrode.

According to some embodiments, the device further comprises a power supply configured to supply a voltage causing the electrical current. In some embodiments, the power supply is configured to supply a voltage causing an electrical current having a frequency in radiofrequency range, optionally in the range of from about 0.2 MHz to about 40 MHz. In some embodiments, the power supply is configured so that the electrical current provides energy in the range of from about 1 to about 200 mJ through each contact surface of each contact electrode.

According to some embodiments, the contact surface of each of contact electrodes has a cross-sectional dimension in the range of from about 25 to about 500 microns.

According to some embodiments, the device is configured to identify a measure of quality of the electrical contact between electrode surfaces and skin surface at a given moment. In some embodiments, the device is configured to adjust parameters of an electric current through the contact surface dependent on the measure of contact quality.

According to some embodiments, the device is configured to measure the impedance of a circuit comprising the contact electrode, the ground electrode, the power supply and the skin. In some embodiments, the device is configured to adjust parameters of an electric current through the contact surfaces depending on the measured impedance.

According to some embodiments, the device comprises a plurality of protruding elements extending from a base section, wherein the contact electrodes are embedded within the protruding elements.

According to some embodiments, the protruding elements comprise substantially parallel, elongated elements.

In some embodiments, a single contact electrode is embedded within a single protruding element. In some embodiments, in a single protruding element are embedded a contact electrode and a ground electrode. In some embodiments, in a single protruding element are embedded at least two ground electrodes and a contact electrode. In some embodiments, the ground electrodes are embedded within the protruding elements.

According to some embodiments, a total contact surface area of contact electrodes is substantially equal to a total surface area of ground electrodes.

According to some embodiments, a total contact surface area of contact electrodes is less than a total surface area of ground electrodes.

According to some embodiments, the device comprises two parts, a first part comprising a lead connected to the power supply and a second part comprising the contact surfaces of contact electrodes, wherein the first part and the second part are reversibly couplable, wherein in at least one coupled state, there is electrical communication between the lead of the first part and the contact surfaces of the second part. In some embodiments, the second part comprising the contact surfaces is disposable.

According to some embodiments, the electrodes comprise an array selected from the group consisting of a one-dimensional array and a two-dimensional array.

According to some embodiments, there is provided a method for stimulation of hair re-growth, the method comprising providing a device for stimulation of hair re-growth on an area of skin of a subject, the device comprising an array of at least one, optionally at least two contact electrodes, configured to conduct electrical current to a surface of an area of skin via a contact surface; contacting the device with the area of skin; and passing an electrical current through the contact surface of the contact electrode, wherein the electrical current delivers energy sufficient to cause a localized heating effect in the area of skin, thereby promoting hair re-growth. In some embodiments, a localized wound is induced, wherein healing of the wound stimulates hair-regrowth. In some embodiments, the device is moved across the area of skin at a speed in the range of from about 0.5 to about 20 cm/sec. In some embodiments, the device comprises an array of at least two contact electrodes.

According to some embodiments, a device used in the method for stimulation of hair growth may comprise any of the devices described herein.

According to some embodiments, the device is configured to conduct electrical current only when in stationary contact with an area of skin (i.e. stamping mode).

According to some embodiments, the device is configured to intermittently conduct electrical current while moving across an area of skin.

Unless otherwise defined, all technical and scientific terms used herein have the same meaning as commonly understood by one of ordinary skill in the art to which the invention pertains. In case of conflict, the specification, including definitions, will control.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Although the invention has been described in conjunction with specific embodiments thereof, it is evident that many alternatives, modifications and variations will be apparent to those skilled in the art. Accordingly, it is intended to embrace all such alternatives, modifications and variations that fall within the scope of the appended claims.

### BRIEF DESCRIPTION OF THE FIGURES

Some embodiments of the invention are described herein with reference to the accompanying figures. The description, together with the figures, makes apparent to a person having ordinary skill in the art how some embodiments of the invention may be practiced. The figures are for the purpose of illustrative discussion and no attempt is made to show structural details of an embodiment in more detail than is necessary for a fundamental understanding of the invention. For the sake of clarity, some objects depicted in the figures are not to scale.

In the Figures:

FIG. 1A is a schematic side view of an embodiment of a device for stimulating hair growth, in accordance with the principles of the present invention;

FIG. 1B is a lower cross-sectional view of the device of FIG. 1A showing a contact electrode embedded in each of the teeth of the device;

FIG. 2A is a schematic side view of an alternative embodiment of the device of the present invention;

FIG. 2B is a lower cross-sectional view of the device of FIG. 2A showing a contact electrode embedded in each of the teeth of the device;

FIG. 3A is a schematic side view of an additional alternative embodiment of the device of the present invention;

FIG. 3B is a lower cross-sectional view of an embodiment of the device of FIG. 3A, showing a contact electrode and a ground electrode of different size embedded in the teeth of the device;

FIG. 3C is a lower cross-sectional view of an embodiment of the device of FIG. 3A, showing a contact electrode and a ground electrode of equal size embedded in the teeth of the device;

FIG. 3D is a lower cross-sectional view of an embodiment of the device of FIG. 3A, showing one contact electrode and two ground electrodes embedded in the teeth of the device;

FIG. 4A is a schematic side view of an additional alternative embodiment of the device of the present invention;

FIG. 4B is a lower cross-sectional view of the device of FIG. 4A showing a contact electrode embedded in each of the teeth of the device

FIG. 4C is a lower cross-sectional view of the device of FIG. 4A showing alternate contact electrodes and ground electrodes embedded in adjacent teeth of the device;

FIGS. 5A and 5B show schematic side views of an additional alternative embodiment of the present invention;

FIG. 5C shows a front view of the device of FIG. 5B;

FIG. 6 shows a perspective front view of an additional alternative embodiment of the present invention;

FIGS. 7A, 7B, and 7C show schematic representations of the device of FIG. 2 a, comprising an array of 5 groups of electrodes; and

FIGS. 8A and 8B are photographs of a patient before and after treatment, respectively, according to the principles of the present invention.

### DETAILED DESCRIPTION OF THE INVENTION

The invention, in some embodiments, relates to the field of cosmetic treatment, and more particularly, but not exclusively, to methods and devices for stimulation of hair growth.

The principles, uses and implementations of the teachings herein may be better understood with reference to the accompanying description and figures. Upon perusal of the description and figures present herein, one skilled in the art is able to implement the invention without undue effort or experimentation. In the figures, like reference numerals refer to like parts throughout.

Before explaining at least one embodiment in detail, it is to be understood that the invention is not necessarily limited in its application to the details of construction and the arrangement of the components and/or methods set forth herein. The invention is capable of other embodiments or of being practiced or carried out in various ways. The phraseology and terminology employed herein are for descriptive purpose and should not be regarded as limiting.

According to some aspects of the present invention, there is provided a method for the cosmetic treatment of hair loss. In some embodiments, the method comprises providing a device for stimulation of hair re-growth on an area of skin of a subject, the device comprising an array of electrodes, each of the electrodes configured to conduct electrical current to a surface of an area of skin which the electrode contacts wherein the electrical current delivers electrical energy sufficient to cause a localized thermal effect at the area of skin; and while moving the device relative to the skin surface intermittently conducting such electrical current to the skin through the electrodes to cause a plurality of localized thermal zones.

As used herein, the term “thermal zone” refers to a zone (volume) of tissue in an area of skin located directly below a contact electrode where upon application of electrical current according to the teachings herein, the tissue temperature is increased to a desired level. In some embodiments a desired level is about 100° C. to cause ablation. In some embodiments, a desired level is in the range 43-100° C. to cause coagulation. In some embodiments, a desired level is below about 43° C. to cause heating without substantial irreversible effects. In some embodiments, ablation and coagulation may be associated with wound creation and wound healing processes. In some embodiments, heating below 43° C. does not introduce wounding but stimulates hair regrowth nevertheless. The level of heating effect is controlled by the level of RF energy delivered to the tissue.

In some embodiments, the localized thermal effect causes at least one localized wound, wherein healing of the localized wound stimulates hair growth.

The parameters of the electric current (e.g., power, frequency, voltage) may be varied so as to maintain a substantially similar level of energy delivered to an area independent of the total surface area of contact surfaces contacting the skin surfaces. As used herein, the term “surface area” refers to an area ordinarily contacted when properly used by a person in accordance with the instructions.

The method described herein may be implemented using any suitable device. In some embodiments it is preferred to use a dermatological treatment device as described herein.

According to some aspects of the present invention, there is provided a device for stimulation of hair re-growth on an area of skin of a subject, the device comprising an array of contact electrodes, each contact electrode being configured to conduct electrical current to a surface of an area of skin via a contact surface. The electrical current delivers electrical energy sufficient to cause a localized thermal effect at the area of skin, thereby stimulating hair re-growth. In some embodiments, the localized thermal effect causes at least one localized wound, wherein healing of the wound stimulates hair re-growth.

The devices and methods of the present invention are applied to the outer layer (epidermis) of the skin, which is less conductive than underlying layers, such that the density of electrical current is relatively high in the epidermis. The outer layer of the epidermis, the stratum corneum, is dielectric and resistant to electrical breakdown up to a certain voltage level. When the voltage exceeds the electrical breakdown threshold, an electrical discharge takes place, which affects the stratum corneum. In some embodiments, the electrical discharge causes shallow wounds.

Preferably, a plurality of small wounds are produced and allowed to heal in an area of skin, rather than damaging a large contiguous area of skin, a technique known as fractional technology. It is believed that the body can more easily heal a small damaged area surrounded by healthy tissue then a large damaged area. The size and depth of the wounds is determined by the size of the electrodes applied to the skin, as well as the specific parameters of the electrical current, such as energy, power, voltage and frequency.

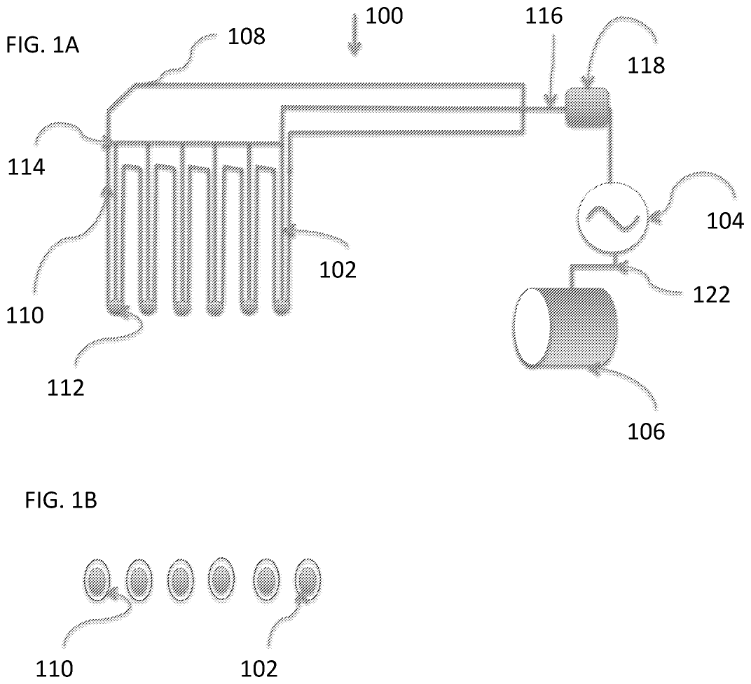

An exemplary device 100, in accordance with the principles of the present invention, is shown in FIG. 1A.

As seen in FIG. 1A, device 100 comprises an array of contact electrodes 102, connected via an alternating current power supply 104 to at least one ground electrode 106, wherein power supply 104 is configured to supply alternating electric current, typically at radiofrequency voltage. Device 100 further comprises a housing 108, having a plurality of protruding elements comprising elongated teeth 110, such that housing 108 resembles, and can function as, a comb. A portion of housing 108 enclosing contact electrodes 102 is fashioned from an insulating material, such as polycarbonate, thereby enabling a user to directly hold housing 108 in the hand during use, while exposing ground electrode 106 to the skin of the user, such that when a user holds device 100, the hand of the user makes electrical contact with ground electrode 106.

Housing 108 is of suitable dimensions to enable device 100 to be comfortably held in the hand of a user during operation. Housing 108 may optionally further be provided with a gripping area or handle (not shown).

Each of contact electrodes 102 is embedded within a distal end of one of elongated, protruding teeth 110, such that a contact surface 112 of each of contact electrodes 102 is exposed. One or more conductors 114, providing electrical contact between power supply 104 through one or more contact electrode leads 116 and contact electrodes 102, is insulatingly embedded within a proximal portion of housing 108.

Contact surfaces 112 are preferably continuously curved, such as, for example, dome-shaped.

Curved surfaces, such as dome shapes, allow reasonable electrical contact with the flexible skin surface without requiring excessive pressure and allow dragging or continuous or stepwise movement across a skin surface (during a combing motion, see below) without causing pain or discomfort.

Elongated teeth 110 are preferably capable of being easily bent without breaking, such that any existing hairs in the treated area are easily separated to enable contact surface 112 to contact the skin surface. The profile of each of elongated teeth 110 of device may be of any suitable shape, such as, for example, circular or elliptical (as in FIG. 1B). Alternatively, elongated teeth 110 may be any suitable cross-sectional shape, preferably of rigidity, dimensions and material similar to those of the teeth of a comb for combing hair

The cross-sectional dimension of contact surface 112, such as the diameter of a contact surface 112 of circular or elliptical teeth 110, influences the size of the thermal zone, such as the size of a wound, induced by each contact surface 112 as well as the parameters of the electrical current required to produce the desired effect. Preferably, the cross-sectional dimension of contact surfaces 112 is in the range of from about 25 to about 500 micrometers, such that the size of a wound is no greater than about 500 micrometers, preferably no greater than about 200 micrometers.

In some embodiments, teeth 110 may be flexible, to provide adequate contact of contact surfaces 112 of contact electrodes 102 with a curved skin surface, such as that of the scalp.

The plurality of teeth 110 is any suitable number of teeth 110 greater than 1, preferably greater than 2. Generally, when the number of teeth 110 is small, providing a narrower array, more parallel strips are needed to treat an area of skin and with a larger number of teeth, (wider array) the device is less maneuverable. Hence, the number of teeth 110 should be sufficient to provide fast and efficient treatment of a skin area without the need for an excessive number of repeated applications, but small enough such that the array is of a width which may be contained within housing 108 of a convenient size to be held easily in the hand of the user. In some embodiments, a center-to-center separation distance for adjacent electrodes is in the range of from about 3 mm to about 4 mm

In some embodiments, contact electrodes 102 are arranged as a one-dimensional array (a line), that in some embodiments is oriented substantially perpendicular to the direction in which the device is moved along the skin surface.

In some embodiments, contact electrodes 102 are arranged as a two-dimensional array, in a manner of a comb or brush.

Two-dimensional arrays may comprise an evenly or randomly spaced matrix, of for example 8×8, 12×12, 16×16, 16×24 electrodes, or any other number and configuration of electrodes.

The distance between any two adjacent contact electrodes 102 is any suitable distance which is generally determined according to clinical factors known in the art of fractional technology, and is sufficient to provide effective hair separation. In some embodiments, the center-to-center separation distance for adjacent contact electrodes 102 is in the range of from about 1 to about 3 mm.

In some embodiments, the alternating voltage causing the electrical current supplied by power supply 104 is in the radiofrequency (RF) range, preferably in the range of from about 0.2 MHz to about 40 MHz, and more preferably in the range of from about 1 MHz to about 15 MHz. The voltage of alternating current is any suitable voltage that is determined by a person having ordinary skill in the art based on factors such as the cross sectional area of the contact electrodes, the extent of wounding desired and safety factors. That said, in some embodiments, the preferred voltage is between 10 V and 400 V, more preferably between 10 V and 150 V. The current power is preferably such that a desired degree of ablation, coagulation or heating of the fraction of the skin occurs.

According to some embodiments, a pulse width of electrical current is selected so as to be short enough to produce small, spaced-apart wounds as device 100 is moved over the skin surface. According to some embodiments, a pulse width of electrical current is in the range of from about 200 μsec to about 500 msec, preferably from about 1 to about 20 msec.

According to some embodiments, all contact electrodes 102 are configured to conduct current simultaneously. According to some embodiments, individual or subgroups of contact electrodes 102 are activated sequentially, in fixed or random sequences, for example, in order to prevent or minimize pain experienced by the user.

In some embodiments, the ratio of the volume of heated to non-heated skin within a treated area is low in order to minimize pain and provide faster and more effective healing. In some embodiments, multiple applications of the device may be required in order to produce the desired effect.

Device 100 further comprises a trigger 118 configured to allow the passage of current through a circuit comprising contact electrodes 102, ground electrodes 106, power supply 104 and a treated skin surface. Trigger 118 of device 100 is an intermittent trigger, which is automatically activated at predetermined intervals of time, e.g., at a rate of 3 to 10 Hz. Trigger 118 is functionally associated with a timer (not shown).

Alternatively, trigger 118 may be activated manually by the user, or may be activated automatically at predetermined distance intervals as device 100 travels over the skin surface. A trigger is typically an assembly of various electric components, in some embodiments including a microprocessor or printed circuit board, functional to trigger the device to conduct an electric current as described herein. In such embodiments, trigger 118 is functionally associated with a distance-measurer (not shown), the distance measurer configured to determine a distance traveled by the device along a skin surface in a prescribed direction when in contact with the skin surface, allowing the electrical current that causes wounds to be supplied at intervals so that the wounds are separated by a desired distance, e.g., between 1 mm and 5 mm. Any suitable distance measurer may be used in implementing the teachings herein, such as, for example, a mechanical rolling component and/or timer component and/or electrooptical component such as used in a computer mouse.

Device 100 is configured to be hand-operable, allowing a user to manually move device 100 along the surface of the skin in a prescribed direction, in a combing motion, analogous to a conventional hair comb. The device is either held by gripping housing 108, or by holding a handle optionally attached to the device (not shown). Current is periodically applied to the skin during motion of the device over the surface of the skin, under control of trigger 118. To treat a large surface area, the device is moved in a prescribed direction to treat a first strip of skin, and then relocated and moved in substantially the same way to treat a following strip of skin parallel to the first strip of skin. The device is preferably moved manually along the skin surface by a user, thus avoiding the need for complex control systems.

Device 100 may optionally be configured to conduct electrical current only when in stationary contact with an area of skin, or to conduct electrical current intermittently while moving across an area of skin.

A challenge in implementing the teachings herein relates to safety. Generally, a device is configured to conduct an electric current that is sufficient to cause a desired level of heating effect to the skin area. If the electric current is such that the localized heating is insufficient, the use of the device is likely ineffective. If the electric current is such that heating is too strong, the use of the device is potentially painful or even damaging to the skin. As long as all contact surfaces are in contact with a skin surface, and therefore the total area of contact surfaces in contact with a skin surface is known, a certain predetermined current with specific parameters can be conducted to cause the desired level of heating. However, if not all the contact surfaces are in contact with the skin surface, the total area of contact surfaces in contact with the skin surface is relatively small; if the certain predetermined current is conducted through the relatively small total area, the damage caused at the contacting surfaces may be too strong.

Accordingly, in some embodiments, the device is configured to identify a measure of the total surface area of contact surfaces (typically, the number of contact surfaces) in contact with a skin surface at given moment, typically just before or during the conducting of the electric current.

In some embodiments, the device is configured to identify a measure of the quality of the contact between the surfaces of the electrodes and the skin surface at a given moment.

In some embodiments, the device is configured to adjust the parameters of an electric current through the contact surfaces dependent on the measure of contact quality.

Any suitable method, component or assembly may be used to identify a measure of the total surface area of contact surfaces in contact with the skin surface at a given moment, according to the total number of electrodes present.

In some embodiments, the device is configured to determine the impedance of a circuit comprising the contact electrodes, the ground electrode and a voltage or current supplies a measure of the total surface area of contact surfaces in contact with a skin surface.

In some embodiments, the device is configured to measure the impedance from contact electrodes 104, through a skin surface, body and to ground electrode 106. In some embodiments, the impedance is measured immediately prior to the supplying of the electrical current. For example, in some embodiments, each event of supplying electrical current is immediately (typically, within 100 milliseconds) preceded by a non-wounding electrical current (e.g., having a potential too low to cause wounds) that gives a measure of the impedance. The parameters of the following electrical current to provide a desired clinical effect are modified as a function of the thus-measured impedance. In particular, in some embodiments when the measured impedance is indicative of lack of contact with a surface, no current is supplied, even when trigger 118 indicates that a current should be applied. In such embodiments, the impedance measuring functions as a contact detector and switch, allowing supply of electrical current only when there is contact with a skin surface.

Some embodiments of a device as described herein comprise a single electric circuit comprising all of the contact electrodes, all of the ground electrodes and the power supply. In some embodiments, a device as described herein comprise at least two electric circuits, each electric circuit including a power supply, at least one contact electrode and at least one ground electrode. In such embodiments the parameters of the current supplied by a power supply of a specific electronic circuit when all electrodes of that circuit contact a skin surface (i.e., all of the contact electrodes and, in some embodiments, also the ground electrodes) are such that a desired level of localized heating is caused.

The shape of the heated zones may be any shape, wherein the width of a zone is defined by the dimensions of contact surface 112, and the length defined by pulse width and speed of movement of the device over the skin surface. In some embodiments, the wound is elliptical.

Device 100 is preferably moved across the skin surface at a speed in the range of from about 0.5 to about 20 cm/sec, more preferably from about 1 to about 10 cm/sec, and most preferably at about 5 cm/sec. According to one non-limiting example, using a contact electrode 102 of power 1 W, with pulse width of about 10 msec and speed of movement of 5 cm/sec, a wound of 500 micrometers length is produced per contact electrode and energy delivered to wound area is 10 mJ.

During use of device 100, ground electrode 106 is in electrical contact with a surface of the person being treated. Ground electrode 106 is of any suitable shape and form. In FIG. 1A, ground electrode 106 is provided in a wrist band 120, connected to power supply 104 via ground electrode lead 122. Alternatively, ground electrode 106 may be provided, for example, on an alligator clip, adhesive pad, or the like. As is clear to a person having ordinary skill in the art, the surface area of ground electrode 106 is substantially greater than the sum of contact surfaces 112 of contact electrodes 102, thereby preventing damage to be caused by the current to a skin surface in contact with ground electrode 106.

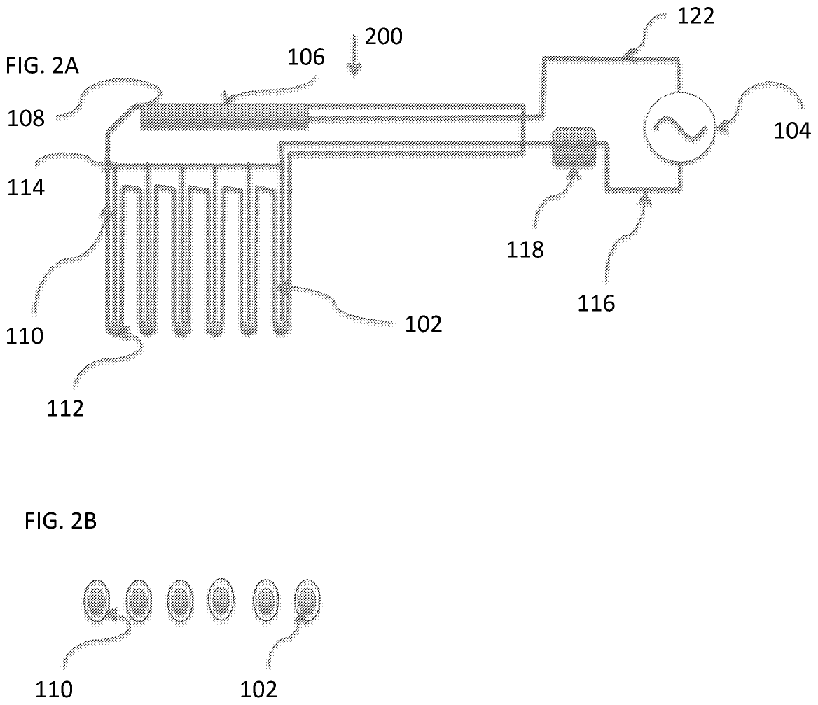

FIG. 2A shows an alternative embodiment 200 of the device of the present invention. Device 200 is similar to device 100 of FIG. 1A, but with ground electrode 106 provided within housing 108, and connected to power supply 104 via ground electrode lead 122. As shown in FIG. 2B, as for FIG. 1B, a contact electrode 102 is embedded in each of teeth 110. Such embodiments are particularly suitable for self-treatment by a user, wherein ground electrode 106 contacts the hand of the user.

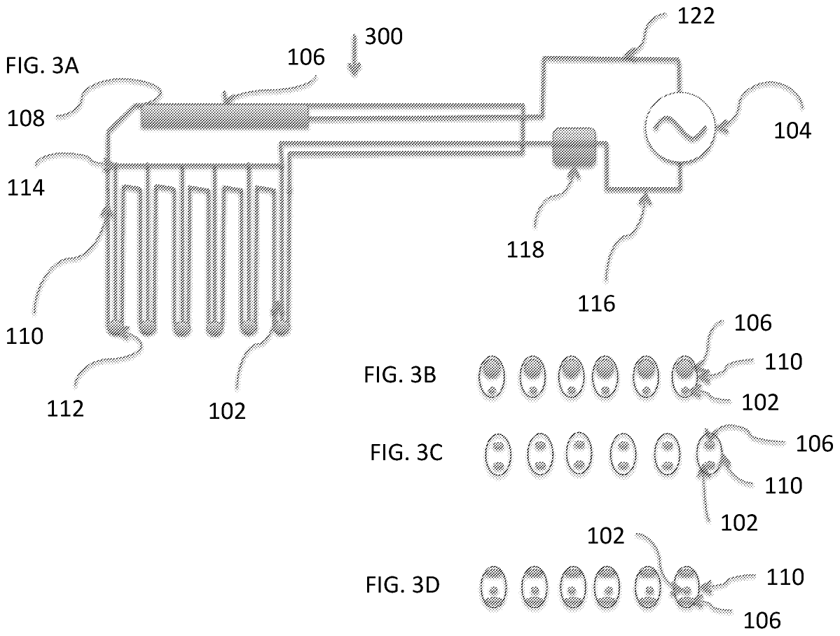

FIG. 3A shows a cross-sectional view of an alternative embodiment 300 of the device of the present invention, wherein a contact electrode 102 and a ground electrode 106, are both embedded within each of teeth 110. In some embodiments, as shown in FIG. 3B, the surface area of a ground electrode 106 is substantially larger than that of a contact electrode 102 so that the electrical current causes skin heating only in proximity of a contact electrode 102. In some embodiments, as shown in FIG. 3C, the surface area of a ground electrode 106 is similar or substantially the same as that of a contact electrode 102 so that the electrical current causes skin heating in proximity of both a contact electrode 102 and a ground electrode 106. In some embodiments, as shown in FIG. 3D, contact electrode 102, and at least two ground electrodes 106 are embedded within each of teeth 110. This configuration provides good contact with contact electrodes 102, even when the user tilts device 300 forward or backward during use. The electrical current heats the skin either at only contact electrode 102 or also at ground electrodes 106, depending on the relative surface areas.

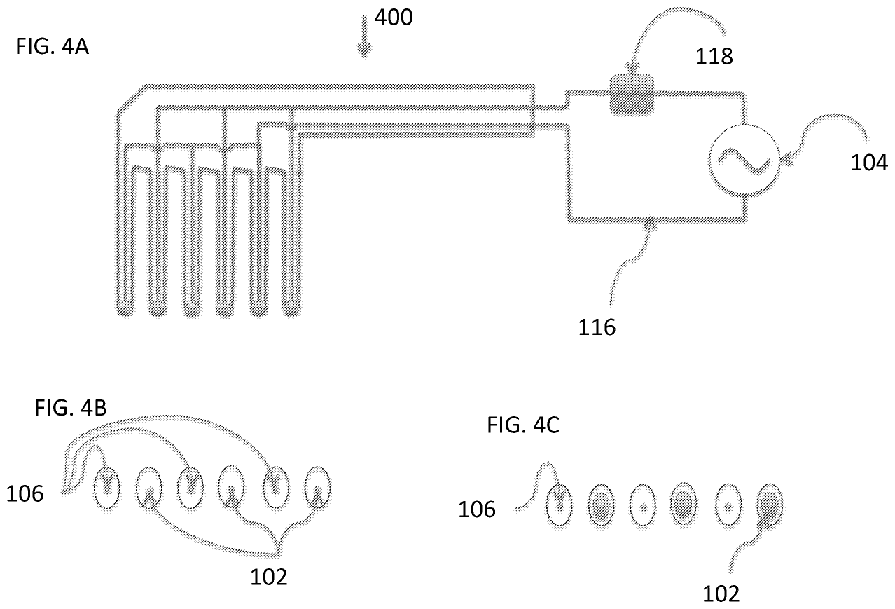

FIG. 4A shows a cross-sectional view of an alternative embodiment 400 of the device of the present invention, wherein adjacent teeth 110 are provided with electrodes of alternating polarity, such that a first of teeth 110 is provided with a contact electrode 102, a second of teeth 110 is provided with a ground electrode 106, a third of teeth 110 is provided with a contact electrode 102, and so on. Analogous to device 300, the electrical current causes skin heating at only contact electrode 102 or also at ground electrodes 106, depending on the relative surface areas.

In some embodiments, the combined surface area of one or more ground electrodes 106 is substantially greater than the combined surface area of contact electrodes 102. In such embodiments, skin heating is caused only by contact electrodes 102. In some such embodiments, ground electrode 106 is remote from contact electrodes 102. For example, ground electrode 106 may be located in a wrist band, patch, or the like, or in an extension of housing 108, such as a handle.

FIG. 4B shows a lower cross-sectional view of the device of FIG. 4A showing a contact electrode embedded in each of the teeth of the device.

FIG. 4C shows a lower cross-sectional view of the device of FIG. 4A showing alternate contact electrodes and ground electrodes embedded in adjacent teeth of the device.

In some embodiments, the combined surface area of one or more ground electrodes 106 is similar or even equal to the combined surface area of contact electrodes 102. In some embodiments, wounds are caused by both the contact electrodes 102 and the ground electrodes 106. In some such embodiments, ground electrodes 106 as well as contact electrodes 102 are embedded within teeth 110. In such embodiments, the path of the electrical current is well defined in the vicinity of the contact surfaces.

In some embodiments, the at least one ground electrode 106 comprises an array of electrodes located proximal to contact electrodes 102.

According to some embodiments, any of the device of the present invention may be used either in stamping mode (i.e., repeatedly relocated to abutting skin areas and triggered), or in moving mode (i.e. triggered upon travelling a pre-defined distance along the skin surface, or at pre-defined time intervals, substantially as described above). In some embodiments, the two-dimensional array of electrodes may comprise two groups comprising electrodes of opposite polarities (i.e. contact electrodes 102 and ground electrodes 106, respectively). In some embodiments, each of the groups may have equal numbers of electrodes 102 and 106. Alternatively, in some embodiments, the number of ground electrodes 106 may be greater than the number of contact electrodes.

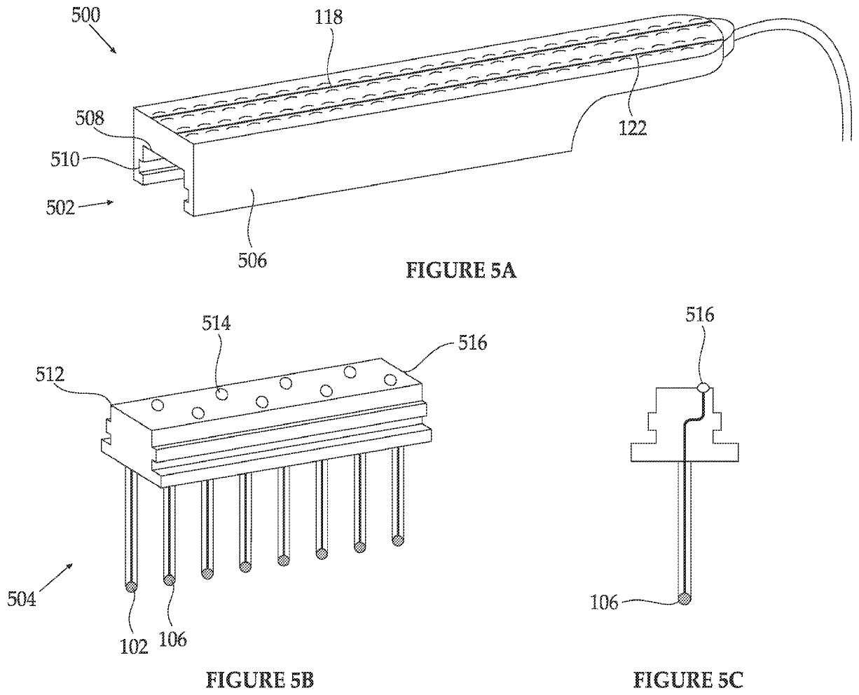

FIGS. 5A-5C show an exemplary device 500 comprising an upper first part 502 and a lower second part 504, parts 502 and 504 being reversibly couplable. First part 502 comprises an elongated member 506 formed from an insulating material, within which are housed contact electrode lead 118 and one or more ground electrode leads 122. Elongated member 506 is formed with a downwardly-facing longitudinal slot 508 along the length thereof, with two longitudinal grooves 510 on either side, wherein leads 118 and 122 are exposed on the upper surface of slot 508.

Second part 504 is formed with a rail 512 configured to slidingly engage slot 508 and grooves 510 of upper part 502. Second part 504 further comprises teeth 110, downwardly extending from rail 512, within which are alternatingly embedded contact electrodes 102 and 106. Conducting contacts 514 and 516 are provided at the upper surface of rail 512, to provide contact between contact electrodes 102 and contact electrode lead 118, and ground electrode 106 and ground electrode lead 122, respectively, when second part 504 is engaged within first part 502.

FIG. 5C shows a head-on view of second part 504, showing a ground electrode 106. As shown, conducting contact 516 for ground electrode 106 is positioned to side of rail 512, such that when second part 504 is engaged within first part 502, conducting contact 516 is in contact with ground electrode lead 122. Conducting contact 514 for contact electrode 102 is positioned at the other side of rail 512 (not shown), such that when second part 504 is engaged within first part 502, conducting contact 514 is in contact with ground electrode lead 118.

In some embodiments, second part 504, including electrodes 102 and 106 is disposable.

In some embodiments, device 500 comprises a number of interchangeable options for second part 504, for example, varying in number and/or arrangement and/or inter-electrode distances of contact electrodes 102 and/or ground electrodes 122, differently-shaped contact surfaces, differently-sized contact surfaces such that one or more users may each select a suitable treatment.

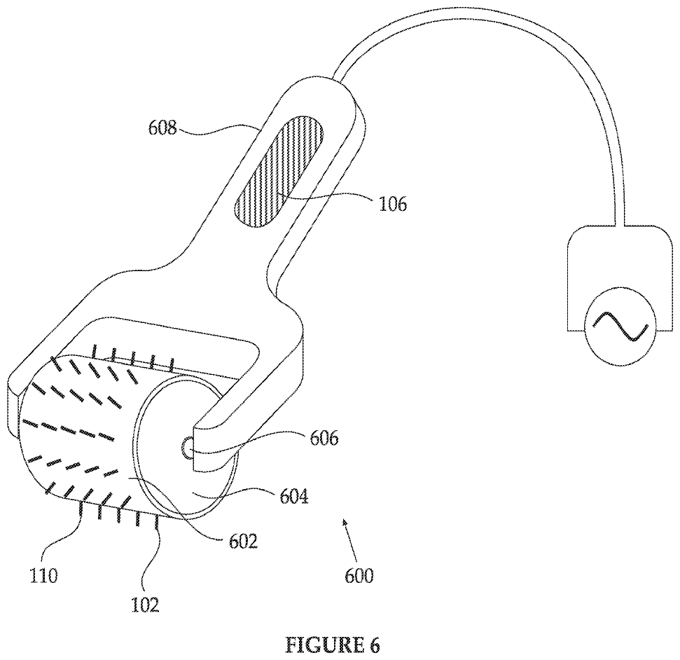

FIG. 6 shows device 600 wherein a plurality of elongated teeth 110 are arranged so as to extend outwards from the outer surface 602 of a cylindrical roller 604. Each of a plurality of contact electrodes 102, each having a contact surface 106 are embedded in one of elongated teeth 110 such that contact surface 106 is exposed. Cylindrical roller 602 is arranged around a rotation shaft 606. A handle 608 constructed from an insulating material extends from both ends of rotation shaft 606, enabling device 600 to be easily held by a user during operation. Embedded inside handle 608 and exposed for electrical contact with a hand holding handle 608 is ground electrode 106. Contact electrodes 102 are connected via a power supply 104 to ground electrode 106. As described above, power supply 104 is configured to supply electric current.

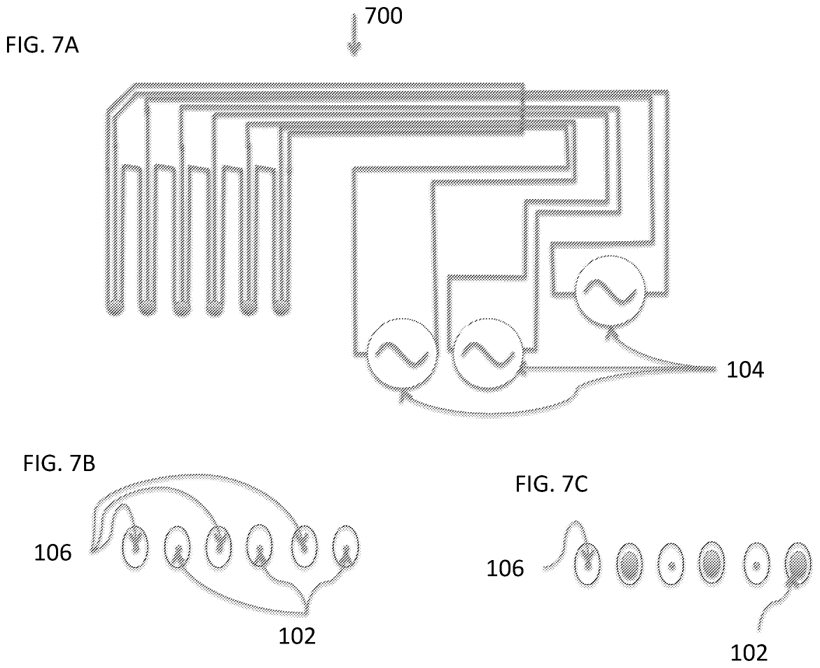

FIG. 7 shows is a schematic representation of operation of device 200 having a power supply 104, an array of electrodes 1, 2, 3, 4, 5, and a switching mechanism 700. Switching mechanism 700 enables the function of each of the five electrodes to independently function as either a contact electrode or a ground electrode. In the example of FIG. 7, switching mechanism 700 is connected in such a way that electrodes 1-4 function as ground electrodes, and electrode 5 as a contact electrode.

### Example a Pilot Study on Hair Growth Stimulation was Carried Out as Follows: Method

A test group of eight patients participated in the pilot study: five females aged 39 to 73 with moderate to severe female pattern hair loss, and three males aged 38 to 50 with male pattern hair loss.

All patients (females and males) had previously used Minoxidil® without any success and two of the three male patients had also previously used Finasteride® without any success.

The Ludwig classification of the female patients was II (moderate)-III (extensive). The Norwood classification of the male patients was IV.

Areas of hair loss on the scalp of the subjects were treated using a device substantially similar to that of FIG. 4c as described above, at frequency 1 MHz every two weeks for a period of at least 8 weeks. The device was used in stamping mode, with treatment applied to successive 1.5 cm2 areas of skin.

The patients were monitored clinically and photographically and were asked to report on any side effects. Dermatoscopic photographs were also taken at each visit. Patient assessment and satisfaction was rated using the GAIS (Global Aesthetic Improvement Scale) scale after the 4th and the 8th treatment. In this scale, grade 1 indicates exceptional improvement, grade 2 indicates significant improvement, grade 3 indicates moderate improvement, grade 4 indicates no improvement, and grade 5 indicates worsening of the condition.

### Results

Seven of the eight patients have been treated for more than half a year (more than 12 treatments) and one male patient has received five treatments. One patient has been treated for nearly a year

All patients show a remarkable improvement. All patients reported arrest of hair growth, and faster hair growth than before commencement of treatment, as identified by more frequent requirement to visit the hairdresser. All had fuller and more aesthetic hair. In six out of eight patients, many new hair follicles were seen in the treated area after 4-8 treatments.

Patient satisfaction was rated after the 4th and the 8th treatment: Five out of eight patients reported exceptional improvement (GAIS grade 1) and three out of eight reported significant improvement (GAIS grade 2) and were very satisfied with the treatment.

The treatment was reported as being painless, and no side effects were seen. Mild erythema was seen in some patients at the treated area directly after the treatment, which lasted only up to a few hours after the treatment.

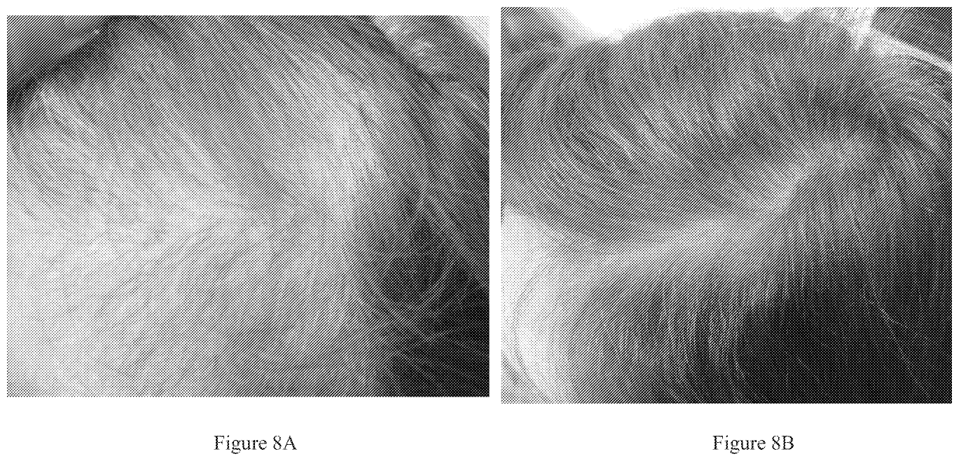

An exemplary photograph of a patient is shown in FIG. 8, before treatment (FIG. 8A) and after treatment (FIG. 8B).

In some embodiments, for example of any of the devices of the present invention as described above, an electrical pulse is triggered every time the device travels a prescribed distance relative to the skin surface. The prescribed distance can be any suitable prescribed distance. In some embodiments, the prescribed distance is not more than about 5 mm, not more than about 4 mm and in some embodiments not more than about 3 mm. In some embodiments, the prescribed distance is not less than about 0.5 mm and in some embodiments not less than about 0.9 mm. In some embodiments, the prescribed distance is between about 1 mm and about 3 mm. When the prescribed distance is smaller, triggerings are closer together while when the prescribed distance is greater, triggerings are further apart.

In embodiments wherein electrical pulse is triggered as a function of distance moved, the method is substantially independent of the speed at which the device is moved. As a result, a user may move the device as convenient, with no need for maintaining a constant speed and may change the speed, for example, to maneuver the device around anatomical features.

It is known in the art to stimulate hair growth by exposing a skin surface to coherent or non-coherent light. In some embodiments of a method as described herein, a skin surface is concurrently or simultaneously irradiated with coherent or non-coherent light to stimulate hair growth together with the teachings described herein. According to some embodiments, any of the devices described herein may further comprise a suitable light source, to implement concurrent or simultaneous irradiation with a coherent or non-coherent light source. Examples of suitable light sources include a laser source, preferably a low level laser light source, for example, a commercially available LED or low power semiconductor laser.

As used herein, the terms “comprising”, “including”, “having” and grammatical variants thereof are to be taken as specifying the stated features, integers, steps or components but do not preclude the addition of one or more additional features, integers, steps, components or groups thereof. These terms encompass the terms “consisting of” and “consisting essentially of”.

As used herein, the indefinite articles “a” and “an” mean “at least one” “one or more” unless the context clearly dictates otherwise

A comb-like device for stimulation of hair re-growth on the epidermis of an area of skin of a subject, wherein the device comprising: at least one base section; an array of electrodes comprising at least two contact electrodes, each of said at least two contact electrodes having a continuously curved contact surface, and at least one ground electrode, each of said at least two contact electrodes configured to conduct electrical current to the epidermis of said area of skin via said continuously curved contact surface; said electrical current is configured to deliver energy sufficient to cause localized heating of said epidermis of said area of skin; said localized heating is configured to cause at least one localized wound with size of no greater than about 500 micrometers on said epidermis; a total contact surface area of said at least two contact electrodes is less than a total surface area of said at least one ground electrode; individual or subgroups of said contact electrodes are activated sequentially, in fixed or random sequences, whereby total average power provided by said device is equal to the number of said activated contact electrodes times an average power in the range of about 50 mW to about 500 mW, either per (i) said activated contact electrode, or (ii) per said activated first contact surface; more than two elongated protruding elements extending from said at least one base section, said more than two elongated protruding elements arranged as a line, said at least one base section and said more than two elongated protruding elements configured to function as a comb, each of said more than two elongated protruding elements having embedded within one of said array of electrodes; each said continuously curved contact surface being exposed at a distal end of one of said more than two elongated protruding elements; said elongated protruding elements are of rigidity and dimensions to allow flexible bending of said protruding elements sufficient to provide adequate contact of said continuously curved contact surfaces with a curved skin surface, such as that of a scalp; a means configured to measure a quality of contact between said at least two contact electrodes and said area of skin, said electrical current is adjusted depending on said quality of contact; a contact surface area of a continuously curved contact surface of each of said more than two elongated protruding elements has a cross-sectional dimension in a range from about 25 micrometers to about 500 micrometers; and a power supply configured to supply a voltage causing said electrical current, said voltage being in a range of between about 10 V to about 400 V; said power supply is configured so that said electrical current provides energy in a range from about 1 mJ to about 200 mJ through each said contact surface of each of said at least two contact electrodes.

It is in the scope of the invention wherein said power supply is configured to supply said voltage causing said electrical current having a frequency in radiofrequency range; wherein said frequency is in a range from about 0.2 MHz to about 40 MHz; and/or wherein said power supply is configured so that said electrical current provides energy in a range from about 1 mJ to about 200 mJ through each said contact surface of each of said at least two contact electrodes.

It is also in the scope of the invention wherein the device is configured such that a function of at least one electrode of said array of electrodes can be optionally switched between being a ground electrode and being a contact electrode; wherein said device is configured to adjust parameters of an electric current through said contact surface depending on a measured impedance; and/or wherein a single one of said at least two contact electrodes or a single one of said at least one ground electrode is embedded within a single one of said more than two elongated protruding elements.

It is also in the scope of the invention wherein, within a single one of said more than two elongated protruding elements are embedded at least one of said at least two contact electrodes and at least one of said at least one ground electrode, said at least one of said at least one ground electrode being exposed at a distal end single one of said more than two elongated protruding elements; wherein the device comprises at least two parts, a first part comprising a lead connected to said power supply and a second part comprising all said contact surfaces of said at least two contact electrodes, wherein said first part and said second part are reversibly couplable, wherein, in at least one coupled state, there is electrical communication between said lead of said first part and said contact surfaces of said second part; and/or wherein said second part comprising said contact surfaces is disposable.

It is also in the scope of the invention wherein said array of electrodes is repeated to form a two-dimensional array of electrodes; wherein delivery of said electrical current comprises at least one pulse, each said at least one pulse having a width in a range from about 200 μsec to about 500 msec; wherein said more than two elongated protruding elements are configured to extend outwards from an outer surface of a cylindrical roller, and/or delivery of said electrical current comprises at least one pulse, each said at least one pulse having a width in a range from about 1 msec to about 20 msec.

It is also in the scope of the invention to disclose a comb-like device for stimulation of hair re-growth on the epidermis of an area of skin of a subject. The device comprising: at least one base section; an array of electrodes comprising at least two contact electrodes and at least one ground electrode, each of said at least two contact electrodes configured to conduct electrical current to the epidermis of said area of skin via a continuously curved first contact surface at a distal end of each of said at least two contact electrodes; each of said at least one ground electrode configured to conduct electrical current from said epidermis of said area of skin via a continuously curved second contact surface at a distal end of each of said at least one ground electrode; individual or subgroups of said contact electrodes are activated sequentially, in fixed or random sequences, whereby total average power provided by said device is equal to the number of said activated contact electrodes times an average power in the range of about 50 mW to about 500 mW either (i) per said activated contact electrode, or (ii) per said activated first contact surface; more than two elongated protruding elements extending from said at least one base section, said more than two elongated protruding elements arranged as a line, said at least one base section and said more than two elongated protruding elements configured to function as a comb; at least one member of a group consisting of: one of said at least two contact electrodes, one of said at least one ground electrode and any combination thereof embedded within each of said more than two elongated protruding elements; said more than two elongated protruding elements; each of said first continuously curved contact surfaces being exposed at a distal end of one of said more than two elongated protruding elements and each of said second continuously curved contact surfaces being exposed at a distal end of one of said more than two elongated protruding elements; a means configured to measure a quality of contact between said at least two contact electrodes and said area of skin, wherein said electrical current is adjusted depending on said quality of contact; a contact surface area of a continuously curved contact surface of each of said more than two elongated protruding elements has a cross-sectional dimension in a range from about 25 micrometers to about 500 micrometers; and a power supply configured to supply a voltage causing said electrical current, said voltage being in a range of between about 10 V to about 400 V; said electrical current is configured to deliver energy sufficient to cause localized heating of said epidermis of said area of skin; said localized heating is configured to cause at least one localized wound with size of no greater than about 500 micrometers, a total contact surface area of said at least two contact electrodes is less than a total surface area of said at least one ground electrode; said elongated protruding elements are of rigidity and dimensions to allow flexible bending of said protruding elements sufficient to provide adequate contact of said contact electrodes with a curved skin surface, such as that of the scalp; said power supply is configured so that said electrical current provides energy in a range from about 1 mJ to about 200 mJ through each said contact surface of each of said at least two contact electrodes.

It is also in the scope of the invention wherein the power supply is configured to supply said voltage causing said electrical current having a frequency in radiofrequency range; wherein said frequency is in a range of from about 0.2 MHz to about 40 MHz; wherein said power supply is configured so that said electrical current provides energy in a range of from about 1 mJ to about 200 mJ through each of said first contact surface; and/or wherein it configured such that the function of at least one electrode of said array of electrodes can be optionally switched between being a ground electrode and being a contact electrode.

It is also in the scope of the invention wherein a contact surface of each of said at least two contact electrodes has a cross-sectional dimension in a range of from about 25 micrometers to about 500 micrometers; wherein said device is configured to adjust parameters of an electric current through said contact surface depending on said measured impedance; wherein a single one of said at least two contact electrodes or a single one of said at least one ground electrode is embedded within a single one of said more than two elongated protruding elements; wherein within a single one of said more than two elongated protruding elements are embedded at least one of said at least two contact electrodes and at least one of said at least one ground electrode; and/or wherein the device comprising two parts, a first part comprising a lead connected to said power supply and a second part comprising said contact surfaces of said at least two contact electrodes, wherein said first part and said second part are reversibly couplable, wherein, in at least one coupled state, there is electrical communication between said lead of said first part and said contact surfaces of said second part.

It is also in the scope of the invention the second part comprising said contact surfaces is disposable; wherein said array of electrodes is repeated to form a two-dimensional array; and/or wherein it additionally comprising a trigger configured to conduct said electric current only when in stationary contact with said area of skin.

Citation or identification of any reference in this application shall not be construed as an admission that such reference is available as prior art to the invention.

1. A comb-like device for stimulation of hair re-growth on the epidermis of an area of skin of a subject, wherein the device comprising:
(a) at least one base section;
(b) an array of electrodes comprising at least two contact electrodes, each of said at least two contact electrodes having a continuously curved contact surface, and at least one ground electrode, each of said at least two contact electrodes configured to conduct electrical current to the epidermis of said area of skin via said continuously curved contact surface;
said electrical current is configured to deliver energy sufficient to cause localized heating of said epidermis of said area of skin; said localized heating is configured to cause at least one localized wound with size of no greater than about 500 micrometers on said epidermis;
a total contact surface area of said at least two contact electrodes is less than a total surface area of said at least one ground electrode;
individual or subgroups of said contact electrodes are activated sequentially, in fixed or random sequences, whereby total average power provided by said device is equal to the number of said activated contact electrodes times an average power in the range of about 50 mW to about 500 mW, either per (i) said activated contact electrode, or (ii) per said activated first contact surface;
(c) more than two elongated protruding elements extending from said at least one base section, said more than two elongated protruding elements arranged as a line, said at least one base section and said more than two elongated protruding elements configured to function as a comb, each of said more than two elongated protruding elements having embedded within one of said array of electrodes; each said continuously curved contact surface being exposed at a distal end of one of said more than two elongated protruding elements;
said elongated protruding elements are of rigidity and dimensions to allow flexible bending of said protruding elements sufficient to provide adequate contact of said continuously curved contact surfaces with a curved skin surface, such as that of a scalp;
(d) a means configured to measure a quality of contact between said at least two contact electrodes and said area of skin, said electrical current is adjusted depending on said quality of contact; a contact surface area of a continuously curved contact surface of each of said more than two elongated protruding elements has a cross-sectional dimension in a range from about 25 micrometers to about 500 micrometers; and
(e) a power supply configured to supply a voltage causing said electrical current, said voltage being in a range of between about 10 V to about 400 V;
said power supply is configured so that said electrical current provides energy in a range from about 1 mJ to about 200 mJ through each said contact surface of each of said at least two contact electrodes.

2. The device according to claim 1, wherein at least one of the following is held true:
said power supply is configured to supply said voltage causing said electrical current having a frequency in radiofrequency range;
said frequency is in a range from about 0.2 MHz to about 40 MHz;
delivery of said electrical current comprises at least one pulse, each said at least one pulse having a width in a range from about 200 μsec to about 500 msec; and,
said power supply is configured so that said electrical current provides energy in a range from about 1 mJ to about 200 mJ through each said contact surface of each of said at least two contact electrodes.

3. The device according to claim 1, configured such that a function of at least one electrode of said array of electrodes can be optionally switched between being a ground electrode and being a contact electrode.

4. The device according to claim 1, wherein said device is configured to adjust parameters of an electric current through said contact surface depending on a measured impedance.

5. The device according to claim 1, wherein a single one of said at least two contact electrodes or a single one of said at least one ground electrode is embedded within a single one of said more than two elongated protruding elements.

6. The device according to claim 1, wherein within a single one of said more than two elongated protruding elements are embedded at least one of said at least two contact electrodes and at least one of said at least one ground electrode, said at least one of said at least one ground electrode being exposed at a distal end single one of said more than two elongated protruding elements.

7. The device according to claim 1, comprising two parts, a first part comprising a lead connected to said power supply and a second part, optionally disposal, comprising all said contact surfaces of said at least two contact electrodes, wherein said first part and said second part are reversibly couplable, wherein, in at least one coupled state, there is electrical communication between said lead of said first part and said contact surfaces of said second part.

8. The device according to claim 1, wherein said array of electrodes is repeated to form a two-dimensional array of electrodes.

9. The device according to claim 1, wherein said more than two elongated protruding elements are configured to extend outwards from an outer surface of a cylindrical roller.

10. The device according to claim 1, wherein delivery of said electrical current comprises at least one pulse, each said at least one pulse having a width in a range from about 1 msec to about 20 msec.

11. A comb-like device for stimulation of hair re-growth on the epidermis of an area of skin of a subject, wherein the device comprising:
(a) at least one base section;
(b) an array of electrodes comprising at least two contact electrodes and at least one ground electrode, each of said at least two contact electrodes configured to conduct electrical current to the epidermis of said area of skin via a continuously curved first contact surface at a distal end of each of said at least two contact electrodes; each of said at least one ground electrode configured to conduct electrical current from said epidermis of said area of skin via a continuously curved second contact surface at a distal end of each of said at least one ground electrode;
individual or subgroups of said contact electrodes are activated sequentially, in fixed or random sequences, whereby total average power provided by said device is equal to the number of said activated contact electrodes times an average power in the range of about 50 mW to about 500 mW either (i) per said activated contact electrode, or (ii) per said activated first contact surface;
(c) more than two elongated protruding elements extending from said at least one base section, said more than two elongated protruding elements arranged as a line, said at least one base section and said more than two elongated protruding elements configured to function as a comb; at least one member of a group consisting of: one of said at least two contact electrodes, one of said at least one ground electrode and any combination thereof embedded within each of said more than two elongated protruding elements; said more than two elongated protruding elements; each of said first continuously curved contact surfaces being exposed at a distal end of one of said more than two elongated protruding elements and each of said second continuously curved contact surfaces being exposed at a distal end of one of said more than two elongated protruding elements;
(d) a means configured to measure a quality of contact between said at least two contact electrodes and said area of skin, wherein said electrical current is adjusted depending on said quality of contact;
a contact surface area of a continuously curved contact surface of each of said more than two elongated protruding elements has a cross-sectional dimension in a range from about 25 micrometers to about 500 micrometers; and
(e) a power supply configured to supply a voltage causing said electrical current, said voltage being in a range of between about 10 V to about 400 V;
said electrical current is configured to deliver energy sufficient to cause localized heating of said epidermis of said area of skin; said localized heating is configured to cause at least one localized wound with size of no greater than about 500 micrometers,
a total contact surface area of said at least two contact electrodes is less than a total surface area of said at least one ground electrode;
said elongated protruding elements are of rigidity and dimensions to allow flexible bending of said protruding elements sufficient to provide adequate contact of said contact electrodes with a curved skin surface, such as that of the scalp;
said power supply is configured so that said electrical current provides energy in a range from about 1 mJ to about 200 mJ through each said contact surface of each of said at least two contact electrodes.

12. The device according to claim 11, wherein said power supply is configured to supply said voltage causing said electrical current having a frequency in radiofrequency range.

13. The device according to claim 11, wherein at least one of the following is held true:
said frequency is in a range of from about 0.2 MHz to about 40 MHz;
said power supply is configured so that said electrical current provides energy in a range of from about 1 mJ to about 200 mJ through each of said first contact surface; and,
a contact surface of each of said at least two contact electrodes has a cross-sectional dimension in a range of from about 25 micrometers to about 500 micrometers.

14. The device according to claim 11, configured such that the function of at least one electrode of said array of electrodes can be optionally switched between being a ground electrode and being a contact electrode.

15. The device according to claim 11, wherein said device is configured to adjust parameters of an electric current through said contact surface depending on said measured impedance.

16. The device according to claim 11, wherein a single one of said at least two contact electrodes or a single one of said at least one ground electrode is embedded within a single one of said more than two elongated protruding elements.

17. The device according to claim 11, wherein within a single one of said more than two elongated protruding elements are embedded at least one of said at least two contact electrodes and at least one of said at least one ground electrode.

18. The device according to claim 11, comprising two parts, a first part comprising a lead connected to said power supply and a second part, optionally disposable, comprising said contact surfaces of said at least two contact electrodes, wherein said first part and said second part are reversibly couplable, wherein, in at least one coupled state, there is electrical communication between said lead of said first part and said contact surfaces of said second part.

19. The device according to claim 15, wherein said array of electrodes is repeated to form a two-dimensional array.

20. The device according to claim 15, additionally comprising a trigger configured to conduct said electric current only when in stationary contact with said area of skin.
